(19) United States
(12) Patent Application Publication
(10) Pub. No.: US 2005/0037987 A1
(43) Pub. Date: Feb. 17, 2005

(54) Methods of identifying kinases and uses thereof

(76) Inventors: Cristina M. Rondinone, Libertyville, IL (US); Regina M. Reilly, Libertyville, IL (US); Jill E. Clampit, Burlington, WI (US); Deanna L. Haasch, Grayslake, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 10/829,512

(22) Filed: Apr. 22, 2004

Related U.S. Application Data
(60) Provisional application No. 60/470,647, filed on May 15, 2003.

(51) Int. Cl. A61K 48/00; A61K 38/00; C12N 5/08

(52) U.S. Cl. 514/44; 514/12; 435/370

(57) ABSTRACT

The subject invention relates to a method of identifying kinases as targets which may be utilized in the development of therapies for metabolic disorders. Additionally, the present invention relates to a method of screening potential therapeutic agents for the ability to prevent IRS-1 degradation and enhance insulin signaling.

The present application claims priority to U.S. provisional patent application Ser. No. 60/470,647, filed on May 15, 2003, hereby incorporated in its entirety by reference.

### BACKGROUND OF THE INVENTION

1. Technical Field

The subject invention relates to a method of identifying kinases as targets and to uses of these targets. For example, the targets may be utilized in the development of therapies for metabolic disorders. Additionally, the present invention relates to a method of screening potential therapeutic agents for the ability to prevent IRS-1 degradation and enhance insulin signaling.

2. Background Information

Type 2 diabetes is characterized by abnormalities of insulin secretion and by insulin resistance in the major target tissues producing a diminished uptake and metabolism of glucose. Alterations in the early steps of insulin signaling have been recognized as an important component of many insulin-resistant states (Virkamaki et al., J. Clin. Invest. 103:931-43 (1999)).

Insulin receptor substrate 1 (IRS-1) is an important intracellular molecule that mediates insulin receptor tyrosine kinase signaling, and an IRS-1-related defect may be one of the contributing factors to insulin resistance. Gene disruption of IRS-1 in mice is associated with impaired insulin-stimulated glucose disposal in vivo and glucose transport in vitro (Araki et al., Nature 372:186-190 (1994); Tamemoto et al., Nature 372:182-86 (1994)). Furthermore, fat cells from subjects with type 2 diabetes, have an impaired insulin effect. In other words, these fat cells have reduced insulin-induced tyrosine phosphorylation of IRS-1 and reduced expression of IRS-1 protein, insulin-induced PI3 kinase activation and reduced insulin stimulated glucose transport (Rondinone et al., Proc. Natl. Acad. Sci. USA 94:4171-75 (1997)). In addition, decreased IRS-1 protein has been observed in various animal models of insulin resistance (Saad et al., J. Clin. Invest. 90:1839-49 (1992); Kerouz et al., J. Clin. Invest. 100:3164-72 (1997)) and in in vitro models such as tumor necrosis factor treatment of 3T3-L1 cells or chronic stimulation with insulin.

The basic mechanisms for the regulation of IRS-1 protein levels are not clear, but there is evidence that serine/threonine phosphorylation of IRS-1 through a rapamycin-dependent pathway precedes and triggers its degradation by the proteasome (Pederson et al., Diabetes 50(1):24-31 (2001)). Consequently, methods are necessary in order to identify targets or intermediates in this pathway that may be impacted in such a manner as to inhibit or regulate the function thereof and thus impact insulin resistance and diabetes.

All U.S. patents and publications referred to herein are hereby incorporated in their entirety by reference.

### SUMMARY OF THE INVENTION

The present invention encompasses a method of identifying a kinase or phosphatase that degrades insulin receptor substrate 1 (IRS-1) and reduces insulin-induced phosphorylation of protein kinase B (PKB) in an insulin-resistant cell. This method comprises the steps of: a) transfecting a human hepatoma cell with short interfering ribonucleic acid (siRNA) against the kinase or phosphatase for a time and under conditions sufficient for the cell to incorporate the siRNA into its genome; b) adding insulin to the resulting cell of step (a) for a time and under conditions sufficient for the cell to become insulin-resistant; c) lysing the resulting cell of step (b) and separating resulting proteins; and d) determining IRS-1 protein level and phosphorylation of PKB, as compared to that of a cell transfected with control siRNA, an increased amount of IRS-1 and phosphorylated PKB, as compared to said cell with control siRNA, indicating the kinase or phosphatase degrades IRS-1 and decreases phosphorylation of PKB in the insulin-resistant cell. The human hepatoma cell may be, for example, a HepG2 cell. Further, the kinase may be, for example, S6 KB2, IKK2, PKC theta, pim 2, pyruvate dehydrogenase, PKC iota, PKC delta, UDP-N-acetylglucosamine-2-epimerimase/N-acetylmannosamine, CaMKI-like protein, DAPK2, casein kinase 1 delta, casein kinase 1 gamma 3, DCAMKL1, SnK Akin kinase, NP_{—}067675, STK10, MAGUK p55 member 2, oxidative-stress responsiveness 1 (i.e., serine-threonine kinase 25), NP_{—}060189, inositol 1, 3, 4 triphosphate 5-6 kinase, mitogen-activated protein kinase 4, mitogen-activated protein kinase 7, LIM kinase 2 (isoform 2b), phosphorylase kinase alpha 2, salt-inducible protein kinase, Jun kinase 1, 2, dystrophia myotonica protein kinase, CGPK1, MKK6, serine-threonine protein kinase PRP4 homolog, STE-2-like kinase, protein tyrosine kinase 9, P38 delta or adenylate kinase 3 (alpha-like). The phosphatase may be, for example, PTEN. Determination of IRS-1 protein level and phosphorylation of PKB is performed by adding anti-IRS-1 and anti-phospho-PKB antibodies to the IRS-1 and phospho-PKB proteins for a time and under conditions sufficient for IRS-1/anti-IRS-1 antibody and phospho-PKB/anti-phospho-PKB antibody complexes to form and determining presence or absence of complexes as compared to complexes formed from proteins of the cell transfected with the scrambled or control siRNA.

Further, the present invention includes a kinase or phosphatase identified according to the method described above.

Additionally, the present invention includes a method of treating a condition in a mammal characterized by diminished uptake and metabolism of glucose comprising the steps of administering to the mammal siRNA against a kinase or phosphatase, wherein the kinase is, for example, S6 KB2, IKK2, PKC theta, pim 2, pyruvate dehydrogenase, PKC iota, PKC delta, UDP-N acetylglucosamine-2-epimerimase/N-acetylmannosamine, CaMKI-like protein, DAPK2, casein kinase 1 delta, casein kinase 1 gamma 3, DCAMKL1, SnK Akin kinase, NP_{—}067675, STK10, MAGUK p55 member 2, oxidative-stress responsiveness 1, NP_{—}060189, inositol 1, 3, 4 triphosphate 5-6 kinase, mitogen-activated protein kinase 4, mitogen-activated protein kinase 7, LIM kinase 2 (isoform 2b), phosphorylase kinase alpha 2, salt-inducible protein kinase, Jun kinase 1, 2, dystrophia myotonica protein kinase, CGPK1, MKK6, serine-threonine protein kinase PRP4 homolog, STE-2-like kinase, protein tyrosine kinase 9, P38 delta or adenylate kinase 3 (alpha-like), and the phosphatase is, for example, PTEN, in an amount sufficient to effect treatment. The mammal may be, for instance, a human, a domesticated animal or a non-domesticated animal. The condition being treated may be, for example, is diabetes and, more specifically, Type 2 diabetes.

Moreover, the present invention also encompasses a method of identifying a compound which inhibits or negatively alters (e.g., decreases) the function of a kinase, wherein the kinase or causes IRS-1 degradation and reduces insulin signaling in an insulin-resistant cell. This method comprises contacting the test compound with the kinase for a time and under conditions sufficient for complexes to form between the test compound and the kinase, presence of the complexes indicating a compound which inhibits or negatively alters the function of the kinase.

Further, the present invention includes a method of identifying a compound which inhibits or negatively alters the function of a phosphatase, wherein the phosphatase causes IRS-1 degradation and reduces insulin signaling in an insulin-resistant cell. This method comprises contacting the test compound with the phosphatase for a time and under conditions sufficient for complexes to form between the test compound and the phosphatase, presence of the complexes indicating a compound which inhibits or negatively alters the function of the phosphatase.

Additionally, the present invention encompasses a method of reducing or inhibiting IRS-1 degradation and increasing insulin-induced phosphorylation of PKB in a mammal in need of the reduction or inhibition of IRS-1 degradation and increased insulin-induced phosphorylation of PKB. This method comprises administering to the mammal an siRNA against a kinase, wherein the kinase may be, for example, S6 KB2, IKK2, PKC theta, pim 2, pyruvate dehydrogenase, PKC iota, PKC delta, UDP-N-acetylglucosamine-2-epimerimase/N-acetylmannosamine, CaMKI-like protein, DAPK2, casein kinase 1 delta, casein kinase 1 gamma 3, DCAMKL1, SnK Akin kinase, NP_{—}067675, STK10, MAGUK p55 member 2, oxidative-stress responsiveness 1, NP_{—}060189, inositol 1, 3, 4 triphosphate 5-6 kinase, mitogen-activated protein kinase 4, mitogen-activated protein kinase 7, LIM kinase 2 (isoform 2b), phosphorylase kinase alpha 2, salt-inducible protein kinase, Jun kinase 1, 2, dystrophia myotonica protein kinase, CGPK1, MKK6, serine-threonine protein kinase PRP4 homolog, STE-2-like kinase, protein tyrosine kinase 9, P38 delta or adenylate kinase 3 (alpha-like) in an amount sufficient to effect reduction or inhibition of IRS-1 degradation and increased insulin-induced phosphorylation of PKB. Again, the mammal may be, for example, a human, a domesticated animal or a non-domesticated animal.

Also, the present invention includes a method of reducing or inhibiting IRS-1 degradation and increasing insulin-induced phosphorylation of PKB in a mammal in need of the reduction or inhibition of IRS-1 degradation and increased insulin-induced phosphorylation of PKB comprising administering to the mammal an siRNA against a phosphatase, for example, PTEN, in an amount sufficient to effect reduction or inhibition of IRS-1 degradation and increased insulin-induced phosphorylation of PKB. Once again, the mammal may be, for example, a human, a domesticated animal or a non-domesticated animal.

Additionally, the present invention encompasses a method of decreasing (or inhibiting) IRS-1 degradation and increasing insulin-induced phosphorylation of PKB in a mammal in need of the decrease (or inhibition) of IRS-1 degradation and increased insulin-induced phosphorylation of PKB comprising administering to the mammal an agonist of a kinase, in an amount sufficient to effect the reduced IRS-1 degradation and increased insulin-induced phosphorylation of PKB. The kinase may be, for example, AXL, liver phosphofructokinase, death-associated kinase-3, galactokinase 1 or fyn-related kinase. The mammal may be as described above.

Further, the present invention also includes a method of identifying a compound which increases activity of a kinase, wherein activity of the kinase prevents or inhibits IRS-1 degradation. This method comprises contacting the test compound with the kinase for a time and under conditions sufficient for complexes to form between the test compound and the kinase, presence of the complexes indicating a compound which enhances activity of the kinase. The kinase may be, for example, AXL, liver phosphofructokinase, death-associated kinase-3, galactokinase 1 or fyn-related kinase.

### BRIEF DESCRIPTION OF THE DRAWINGS

FIG. 1 illustrates how insulin induces degradation of IRS-1 in HepG2 human hepatoma cells. In particular, the figure establishes that IRS-1 could be degraded in response to insulin in HepG2 cells, and that the presence of high glucose in the media enhanced this process.

FIG. 2 represents the effect of plating densities and transfection reagents on transfection efficiency. In particular, the figure shows that lowering the plating density resulted in a better transfection efficiency as indicated by consistent knockdown of the target p70S6.

FIG. 3 illustrates the dose-dependent downregulation of retinoblastoma protein levels by retinoblastoma (Rb) siRNA. In particular, at a 100 nM concentration, there was an efficient 50%-60% reduction of protein. At 200 nM, most of the siRNAs had some non-specific effects resulting in the downregulation of non-target proteins. Similar results were obtained with the different siRNAs tested. In view of the data, 100 nM was used in the screening assays.

FIG. 4 illustrates that oligo 87 (S6 KB2 siRNA) prevents IRS-1 degradation and enhances insulin-induced PKB phosphorylation. In particular, the figure establishes that insulin induced degradation of IRS-1 in scrambled control (UC)-treated cells, while oligo 87 siRNA and raptor prevented this effect.

FIG. 5 illustrates that reduction of IKK2 inhibits degradation of IRS-1 and enhances insulin-induced phosphorylation of PKB.

FIG. 6 illustrates RT-qPCR analysis of S6 KB2 expression. The mRNA knockdown for this mRNA was approximately 40%. As a control, the knockdown of S6 KB1 and S6KA4. S6 KB2 siRNA did not downregulate these two homologous kinases indicating that the down regulation of S6 KB2 by siRNA was specific.

FIG. 7 illustrates that pim2 siRNA prevents IRS-1 degradation and enhances insulin-induced phosphorylation of PKB and is therefore an interesting target in connection with the treatment of metabolic diseases.

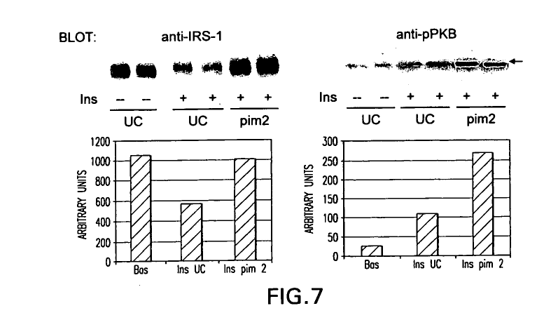

FIG. 8 illustrates RT-qPCR analysis of pim2 expression after transfection with different pim2 siRNAs. The different pim2 siRNAs tested efficiently downregulated pim2 mRNA and did not affect pim1 (data not shown).

FIG. 9 illustrates that reduction of JNK-1 inhibits IRS-1 degradation and enhances insulin-induces PKB phosphorylation. JNK activity is elevated in Type 2 diabetes and insulin resistant states. However, JNK-1−/− mice are protected from the development of diet-induced obesity and insulin-resistance. Reduction of JNK-1 in ob/ob mice improves insulin sensitivity and prevents diabetes.

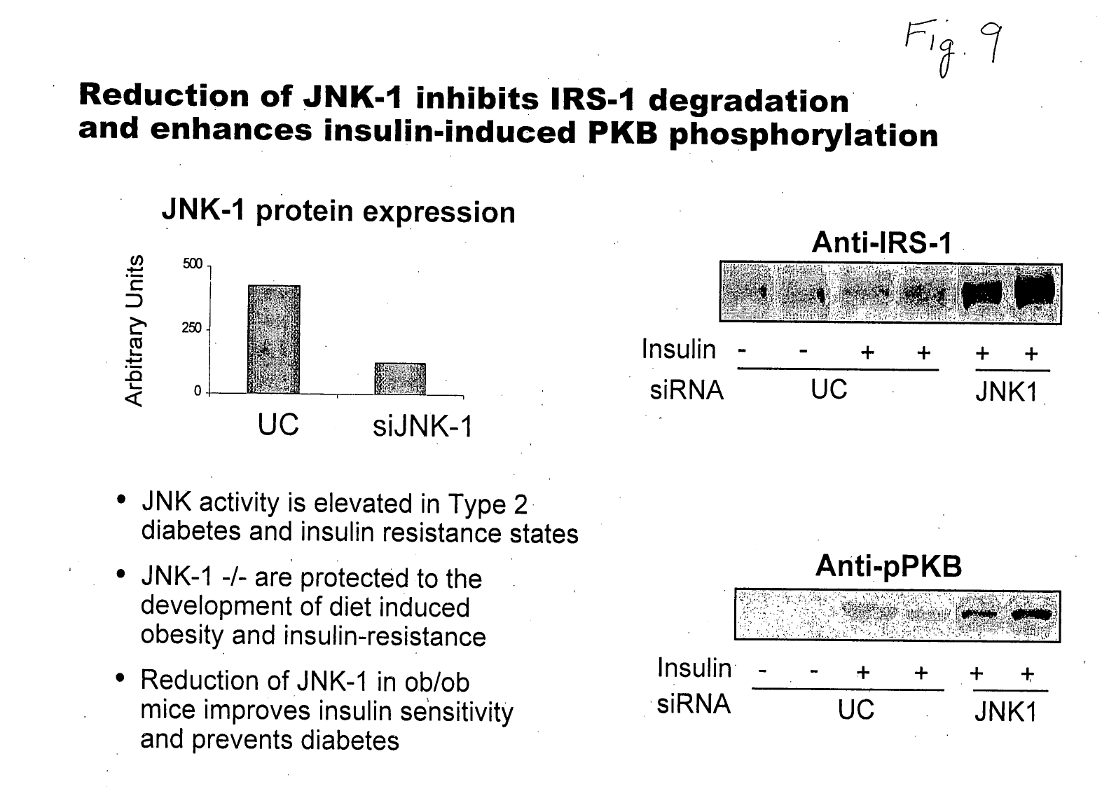

FIG. 10 illustrates that reduction of PKCθ inhibits IRS-1 degradation and enhances insulin-induced PKB phosphorylation. FFA-induced insulin resistance is associated with activation of PKCθ. Further PKCθ knockout mice are protected from lipid-induced insulin-resistance. It should also be noted that increased PKCθ is found in muscle of patients with Type II diabetes, and that chronic hyperinsulinemia increases PKCθ expression.

FIG. 11 provides further data illustrating that reduction of IKK2 inhibits IRS-1 degradation and enhances insulin-induced PKB phosphorylation. Overexpression of IKK2 causes insulin resistance; however, expression of dominant negative IKK2 reverses TNF-, FFA- and hyperglycemia-induced resistance. Heterozygous deletion (IKKβ +/−) protects against development of insulin resistance during high-fat feeding and in obese (ob/ob) mice.

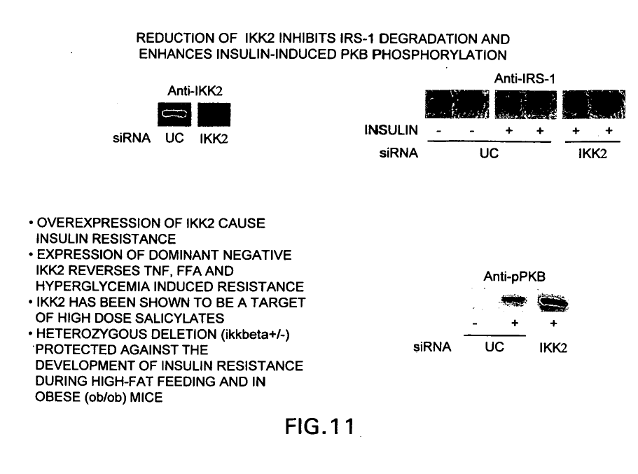

FIG. 12 illustrates the properties of salt-inducible kinase-1 (SIK) which is a novel kinase involved in insulin resistance. SIK was first cloned from the adrenal glands of rats fed a high salt diet. SIK1 mRNA is elevated in adipose tissues, livers and skeletal muscle of diabetic animals. Further, SIK1 is involved in the phosphorylation of Ser⁷⁸⁹ in IRS-1 livers of diabetic animals.

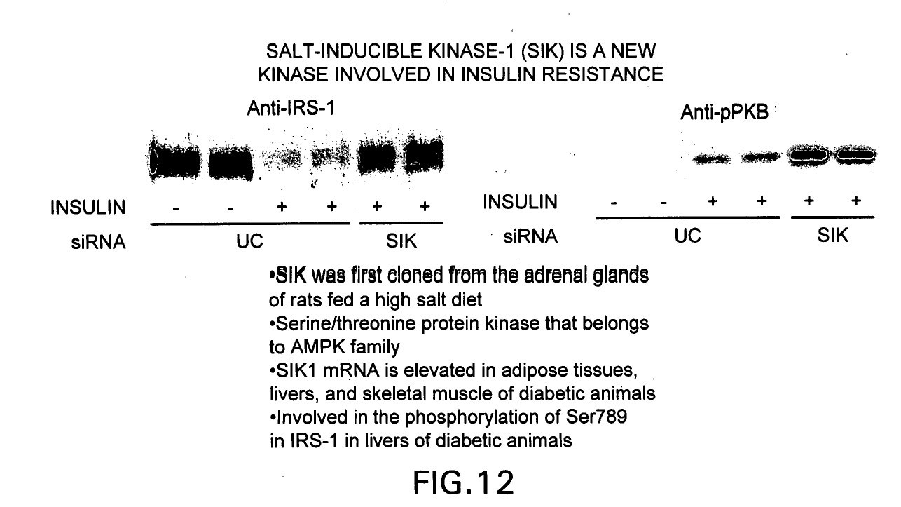

### DETAILED DESCRIPTION OF THE INVENTION

The present invention encompasses a method of identifying new targets (e.g., kinases and phosphatases) for the treatment of disorders associated with insulin resistance as well as methods of screening for agents that prevent IRS-1 degradation and enhance insulin signaling and thus treat type 2 diabetes. Further, the present invention encompasses uses of the identified targets themselves.

Additionally, the present invention encompasses methods of screening for agents, compositions or compounds that induce, enhance or increase activity of a kinase that is known to prevent IRS-1 degradation, and thus increase insulin sensitivity.

At the present time, very few kinases have been identified as targets of insulin resistance and diabetes. Thus, a new method was developed by the present inventors in order to identify potential kinases (and phosphatases as well) involved in insulin resistance. Generally, this method comprises initially transfecting human hepatoma cells (i.e., HepG2 cells) with a kinase or phosphatase-specific siRNA developed against the kinase or phosphatase in question. HepG2 cells are human hepatoma cells that are responsive to insulin. Other suitable cells that may be utilized in the method include primary hepatocytes such as human, rat or mouse hepatocytes. Examples of such siRNAs which may be used to transfect the HepG2 cells include, for example, those against S6BK2, Pim kinases, IKK2, PKC-delta and phosphatidylinositol-3,4,5-triphosphate 3-phosphatase (PTEN). (All of the above, with the exception of PTEN, are kinases. PTEN is a phosphatase which may also be used as a target.)

Subsequent to transfection, the HepG2 cells are incubated with insulin and allowed to develop to an insulin-resistant state (see Example III). An insulin-resistant state is defined as a condition in which there is resistance to the cellular action of insulin. Once this state is achieved, the cells are lysed. The proteins present in the lysates are then separated by gel electrophoresis or any other means utilized to separate proteins which are known to those of ordinary skill in the art (see, e.g., Molecular Cloning: A Laboratory Manual, 2^{nd} edition, 1989, Cold Spring Harbor Laboratory Press, editors J. Sambrook et al.) The separated proteins are then transferred to a membrane such as, for example, nitrocellulose or PDF, and the IRS-1 protein levels and phosphorylation of protein kinase B (PKB) are visualized by, for example, a Western Blot (or any other related visualization means known to those of ordinary skill in the art) using, for example, commercially available antibodies such as anti-IRS-1 and phospho-PKB antibodies.

Such antibodies may be prepared by, for example, injecting the relevant antigen (i.e., IRS-1 or PKB) into a mammal (e.g., a mouse, a rabbit, a dog, etc.), allowing sufficient time for production of an immune response, subsequently injecting the relevant antigen again and harvesting the resulting antibodies from the blood of the mammal.

An increased amount or detection of IRS-1 and phospho-PKB, or phospho-PKB alone, as compared to a control cell (i.e., a cell which has been incubated with insulin and transfected with scrambled siRNA (see Example III)), is indicative of increased insulin sensitivity caused by the siRNA utilized, thereby indicating that the related kinase (i.e., the kinase against which the siRNA is generated) (or phosphatase) is a target which may be used in developing a compound for treatment of a disorder characterized by insulin resistance and diabetes. In particular, a compound may be created which antagonizes or inhibits the function of the kinase (or phosphatase), thereby preventing IRS-1 degradation and enhancing insulin signaling. Thus, until the development of the present invention, the kinases identified herein as targets were not known to be involved in IRS-1 degradation, although some of them were already known to be involved in insulin resistance (see Table I). The role of others had never before been elucidated to any degree.

Additionally, using the identified targets, one may screen for existing compounds which inhibit the function or negatively alter the activity of the target kinase (or phosphatase) of interest. This may be accomplished by exposing the test compound to the kinase (or phosphatase) known to induce IRS-1 degradation and decrease insulin signaling, and determining whether the test compound binds to the kinase (or phosphatase). Such binding, or the formation of complexes, indicates that the test compound is capable of inhibiting or interfering with the action or function of the kinase (or phosphatase) and may therefore be used in vivo to increase IRS-1 levels and insulin signaling in a patient (e.g., a mammal such as a human, domesticated animal, non-domesticated animal, etc.) in need of such treatment.

The present invention may be illustrated by the use of the following non-limiting examples:

### EXAMPLE I

### Identification of a Suitable Cell Line for Transfection and Optimization of siRNA Transfection Conditions

The human hepatoma cell line (HepG2) was initially selected for investigation. This cell line is known to be insulin sensitive; however, it was not known whether insulin could induce degradation of IRS-1 in these hepatocytes, as previously described in fat cells (Sun et al., Diabetes 48:1359-1364, 1999; Pederson et al., Diabetes 50:24-31, 2001).

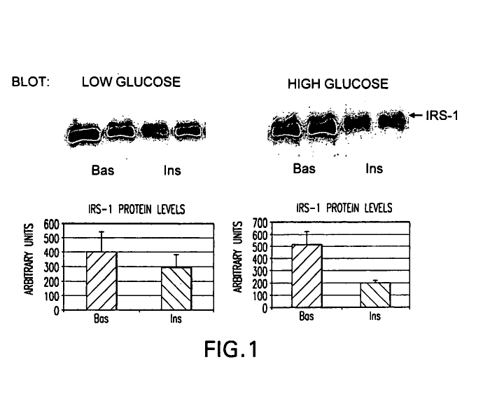

Cells were treated with 1 uM insulin and a high (25 mM) or low (7 mM) concentration of glucose for 18 hours. FIG. 1 shows that IRS-1 could be degraded in response to insulin in HepG2 cells, and that the presence of high glucose in the media enhanced this process. Consequently, the cell line proved to be a suitable candidate for the transfection studies.

Once the cell line was selected, it was important to optimize the siRNA transfection conditions to maintain good transfection efficiency while minimizing non-specific effect. In order to do so, HepG2 cells were plated at different densities on Day 0 in collagen-treated plates so they could grow as a monolayer. On Day 1, cells were transfected with 100 nM of each siRNA oligo, in triplicate wells. The transfection reagent selected was based on an estimate of cell confluence. The polymer-based transfection reagent, Trans-IT TKO (Mirus Corp., Madison, Wis.) reagent protocol suggests approximately 50% confluence, and the Lipofectamine 2000 reagent suggests approximately 90-95% confluence. Thus, Trans-IT TKO worked more efficiently than Lipofectamine 2000 under these conditions. Cells were lysed on Day 3, 48 hours after starting transfection. Following separation of 20 ug total protein on SDS/PAGE gel, the proteins were immunoblotted with anti-p706S kinase or anti-β actin antibody.

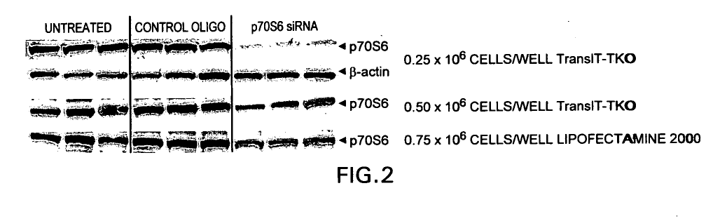

A key factor found to be crucial for efficient transfection was the plating density. In particular, as shown in FIG. 2, lowering the plating density resulted in a better transfection efficiency as indicated by the consistent knockdown of the target p706S kinase.

Further experiments were designed to identify the correct concentration of siRNA to use in the screening assays. In particular, HepG2 cells were seeded in a 6-well collagen-coated plate. After 24 hours, when they reached 30-40% confluency, they were untransfected (Mock) or transfected with several concentrations of different siRNAs including Retinoblastoma (Rb siRNA) or scramble control (UC) siRNA. (For a general discussion of the generation of siRNAs, see Elbashir et al., Genes and Development 15:188-200 (2001); Tuschl et al., Genes and Development 13:3191-3197 (1999); Elbashir et al., Methods 26:199-213 (2002); Elbashir et al., Nature 411:494-498 (2001) and Harborth et al., J. Cell Science 114:4557-4565 (2001).)

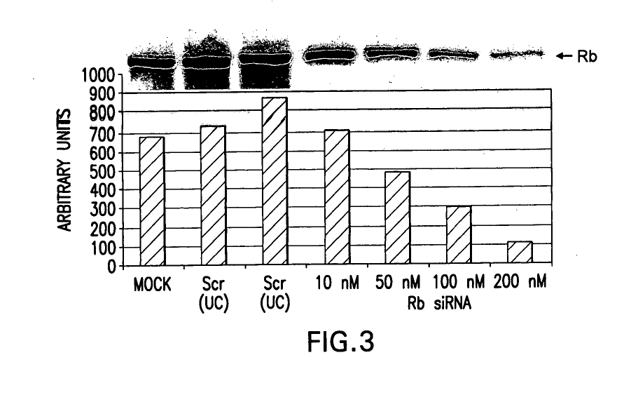

At 48 hours post-transfection, cells were lysed and proteins were separated by SDS-PAGE and immunoblotted using anti-retinoblastoma antibodies. Knockdown of the target kinases was monitored. As FIG. 3 illustrates, there was a dose-dependent effect of the RB siRNAs on the silencing of the retinoblastoma protein levels. At a 100 nM concentration, there was an efficient 50-60% reduction of protein. At 200 nM, most of the siRNAs had non-specific effects resulting in the down regulation of non-target proteins. Similar results were obtained with the different siRNAs tested. Thus, in view of this data, 100 nM were used in the screening assays.

### EXAMPLE II

### siRNA Transfection Protocol

HepG2 cells (available from the American Type Culture Collection, 10801 University Boulevard, Manassas, Va. 20110-2209) were cultured in Minimal Essential Medium (MEM)(#11095, Gibco BRL, Rockville, Md.) containing 1× non-essential amino acids, 1× sodium pyruvate and 10% Fetal Bovine Serum (FBS). Cells were plated at 1.2×10⁵ cells per well in a 6-well collagen-coated plate (BD Biosciences Discovery Labware, Bedford, Mass.) 24 hours prior to transfection. The optimal density for transfection was 30-40% confluency. Transit-TKO (Mirus Corp., Madison, Wis.) was used-as the transfection reagent. Specifically, 8 ul of Transit-TKO was diluted in 400 ul OptiMem (Gibco BRL, Rockville, Md.) and incubated for 10 minutes at room temperature. Seven microliters of 20 uM siRNA (Dharmacon, Lafayette, Colo.) were added to the transfection mix and incubated for an additional 20 minutes at room temperature. The mix was then added to one well of cells, which had been refreshed with 1 ml of culture media. The following day, an additional 1 ml of culture media was added per well. At 48 hours post-transfection, cells were washed once with Phosphate Buffered Saline (PBS) and then incubated with 2 ml of culture media without FBS for 3 hours at 37° C., 5% CO₂. At the time of induction, the media on control cells, not induced by insulin, was changed to MEM/1× non-essential amino acids/1× sodium pyruvate/25 mM glucose/1× amino acids. Cells induced by insulin received MEM/1× non-essential amino acids/1× sodium pyruvate/25 mM glucose/4× amino acids/10 uM human insulin (Sigma, I-9278, St. Louis, Mo.) and incubated for 18 hours at 37° C., 5% CO₂. The cells were then lysed with 200 ul per well of 1× TBS/1% triton X-100, 0.5% NP-40/0.25% sodium deoxycholate/1 mM EDTA/1 mM EGTA/10 mM sodium fluoride/1 mM sodium orthovanadate/1 uM microcystin/1 mM AEBSF/complete EDTA tablet (Roche Indianapolis, Ind.).

### EXAMPLE III

### Identification of Proteins to be Used as Targets

Once the transfection conditions were optimized, a cell-based assay was developed for the screening of 507 kinase-specific siRNAs to determine their: 1) effect on IRS-1 degradation after chronic insulin treatment and 2) enhancement of insulin-induced phosphorylation of PKB.

In particular, in all transfection experiments, HepG2 cells were plated in a 24 well microtiter plate in MEM and transfected with the different kinase-specific siRNAs (100 nM) or scramble control siRNAs for 48 hrs in duplicate samples. At the end of this period, the cells were washed, serum-starved for 3 hrs, and confluent cells were incubated at 37° C. with low (7 mM) or high (25 mM) glucose in the presence or absence of insulin (1 uM). (Presence of the insulin and glucose allowed the cells to develop an insulin-resistant state.) After 18 hours, the cells were lysed and proteins were separated by SDS/PAGE on 7.5% and 10.0% gels. The electrophoresis was run at 120V. The proteins were then transferred from the gel to nitrocellulose sheets and blocked in 5% milk. The blots were then probed with anti-IRS-1 antibodies (Upstate Biotechnology, Lake Placid, N.Y.) or with phosphoserine 473 antibodies (BD Pharmigen, San Diego, Calif.), according to the manufacturer's recommendations. In particular, IRS-1 protein levels and phosphorylation of PKB were visualized by Western Blotting using the anti-IRS-1 and anti-phospho-PKB antibodies. Increased detection of IRS-1 and phospho-PKB, or phospho-PKB only, is indicative of increased insulin sensitivity.

SiRNAs against mTOR-related targets, such as p70S6kinase (i.e., a downstream kinase of mTOR) and raptor, were used in parallel experiments as positive controls. (Raptor is a mTOR binding protein that enhances the mTOR kinase activity toward p70, and inhibition of raptor expression by RNAi reduced mTOR-catalyzed phosphorylation.) The proteins were detected by enhanced chemiluminescence with horse peroxidase-labeled secondary antibodies (Amersham, Piscataway, N.J.). The intensity of the bands was quantitated with a laser densitometer (Molecular Dynamics, Sunnyvale, Calif.).

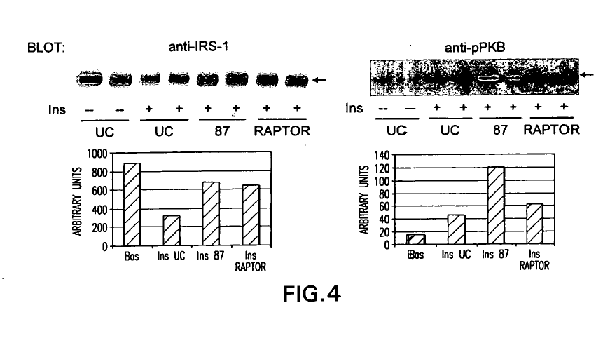

As shown in FIG. 4, insulin-induced degradation of IRS-1 in scrambled control (UC)-treated cells, while oligo 87 siRNA and raptor prevented this effect. Interestingly, oligo 87 increased insulin-stimulated PKB phosphorylation indicating that oligo 87 was a positive hit for the assay. Oligo 87 corresponds to a novel S6K1 homolog, p54 S6 kinase 2 (i.e., p54S6k2/S6 KB2) that is activated by insulin, mitogens and by constitutively active PI3K and is inhibited by rapamycin indicating that this kinase is downstream of mTOR. Thus, this kinase represents an interesting hit in the mTOR pathway inducing insulin resistance. This hit was confirmed in dose-response assays and by evaluating the effectiveness using 2 other S6 KB2 specific siRNAs, as described below.

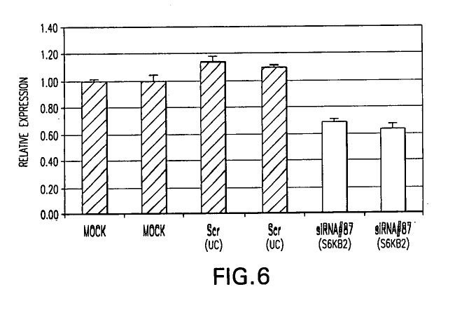

The knockdown of the S6 KB2 mRNA by the siRNA was tested by RT-PCR. As shown in FIG. 6, the mRNA knockdown for this siRNA was approximately 40%. As a control, the knockdowns of S6 KB1 and s6KA4 (S6 kinase homologues) were tested. S6 KB2 siRNA did not downregulate these two homologous kinases indicating that the downregulation of S6 KB2 by siRNA was specific.

Based upon the above screen, several interesting hits have been identified as reflected in Table I below:

TABLE I

| Hits | Comments |
|---|---|
| RPS6KB2 (p70S6 kinase B2 | Kinase downstream mTOR |
| IKK2 | Literature target for diabetes |
| PKC theta | Literature target for diabetes |
| Pim kinases (pim2) | serine/threonine kinase |
| | (proto-oncogene) |
| Piruvate Dehydrogenase kinase | Regulator of gluconeogenesis |
| PKC iota and PKC delta | atypical and novel PKC member |
| UDP-N-acetylglucosamine-2- | expressed mainly in liver; |
| epimerase/N-acetylmannosamine | phosphorylated by PKCs |
| kinase | |
| CaMKI-like protein kinase | calcium calmodulin dependent kinase 1- |
| | like kinase |
| DAPK2 | death associated protein kinase-related |
| | 2 (involved in TNF pathway - |
| | apoptosis) |
| Casein kinase 1 delta, gamma 3 | new members of casein kinase 1 |
| DCAMKL1 | Doublecortin and CaM kinase-like 1 |
| | (unknown function) |
| SnK Akin kinase | serine threonine kinase 18 |
| NP_067675 | human putative GS3955 serine/ |
| (accession number in NCBI | threonine kinase |
| database) | |
| STK10 | serine threonine kinase 10/new polo-like |
| | kinase |
| MAGUK p55, member 2 | membrane-associated guanylate kinase |
| Oxidative-stress responsive 1 | a ser/threonine kinase; activated by |
| (OSR-1) | oxidative stress |
| Serine-threonine kinase 25 | Serine/threonine protein kinase 25 |
| | (Sterile 20/oxidant stress-response |
| | kinase 1) (Ste20/oxidant stress response |
| | kinase-1) (SOK-1) (Ste20-like kinase). |
| NP_060189 | non-fermenting protein (SNF-1)-related |
| (accession number in NCBI | kinases (human; SNRK) |
| database) | |
| Inositol 1, 3, 4 triphosphate 5-6 | |
| kinase | |
| mitogen-activated protein kinase | upstream of JNKs; |
| kinase 4 & 7 | Jun kinase-1 |
| LIM kinase 2, isoform 2b | involved in regulation of cytoskeleton |
| Phosphorylase kinase alpha 2 | Liver |
| SIK | Salt-inducible protein kinase-1 |
| Jun kinases 1, 2 | Jun kinase-1 |
| Dystrophia myotonica protein | |
| kinase | |
| CGPK1 | CGMP-dependent protein kinase type 1 |
| | Crosstalk with insulin signaling |
| MKK6 | MAP kinase kinase kinase 6 |
| | Activates Jun kinases |
| Serine-threonine protein kinase | mRNA Splicing |
| PRP4 homolog | |
| STE-2 like kinase | STE-20 like kinase, activates JNKs |
| Protein tyrosine kinase 9 | Cytoeskeleton movements |
| P38 delta | P38 MAPK |
| Adenylate kinase 3 alpha like | Mitochondrial |

In addition, kinases that were already known to participate in the insulin pathway cascade, leading to IRS-1 degradation, such as PI3-kinase, PDK1, mTOR and p70, were positive hits in the assay.

Table II below illustrates additional data confirming the knockdown of the kinases using siRNA. In particular, HepG2 cells were treated with siRNA at a final concentration of 100 nM for 48 hours in the presence of Transit TKO reagent (Mirus Corp., Madison, Wis.). Reverse transcription and PCR conditions were done at standard temperatures and times using the Invitrogen Platinum Thermoscript One Step System qRTPCR kit (lot# 1188693) (Invitrogen, Carlsbad, Calif.). The comparative Ct (cycle threshold) method was used to determine the fold difference of universal control and siRNA treated cells to mock controls unless noted. The mock control sample RNA was pooled from 3 independent transfections. The data points (run in triplicate assay) were normalized to 28s rRNA. (Template: 100 ng total RNA input/well, (DNAse I treated RNA). The human genes analyzed are as follows:

| Name | Description |
|---|---|
| STK18 | SnK Akin kinase |
| MAGUK p55 | membrane-associated guanylate kinase |
| OSR1 | oxidative stress responsive 1 |
| CSNK1G3 | casein kinase gamma 3 [Homo sapiens]; CK1 gamma 3 |
| SNF-1 | SNF-1 related kinase |
| STK10 | serine/threonine kinase 10 |
| Raptor | raptor; p150 target of rapamycin (TOR)-scaffold |
| | protein containing WD-repeats |
| SIK-1 | salt-inducible kinase 1 |
| GNE | UDP-N-acetylglucosamine-2-epimerase/N- |
| | acetylmannosamine kinase; N-acylmannosamine kinase. |

TABLE II

| Name | Description |
|---|---|
| STK18 | SnK Akin kinase |
| MAGUK p55 | membrane-associated guanylate kinase |
| OSR1 | oxidative stress responsive 1 |
| CSNK1G3 | casein kinase gamma 3 [Homo sapiens]; CK1 gamma 3 |
| SNF-1 | SNF-1 related kinase |
| STK10 | serine/threonine kinase 10 |
| Raptor | raptor; p150 target of rapamycin (TOR)-scaffold |
| | protein containing WD-repeats |
| SIK-1 | salt-inducible kinase 1 |
| GNE | UDP-N-acetylglucosamine-2-epimerase/N- |
| | acetylmannosamine kinase; N-acylmannosamine kinase. |

### EXAMPLE IV

### Effects of IKK2 and PKC Theta Reduction on IRS-1 and PKB

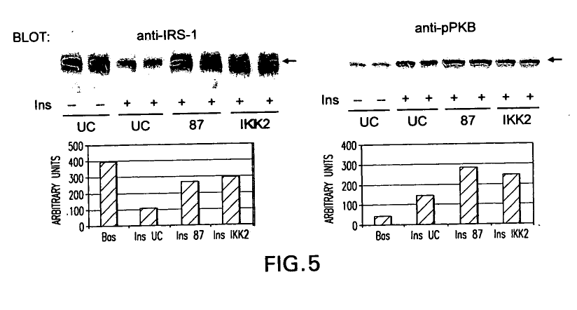

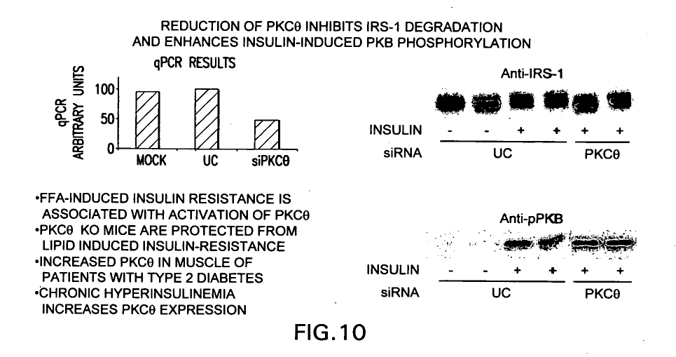

Using the transfection method, two hits were identified referred to as IKK2 and PKC theta. These two kinases caused IRS-1 degradation and reduced insulin-induced phosphorylation of PKB. (See FIG. 5 and FIG. 10.) In the case of PKC theta and IKK2, this data further validates these literature targets. In particular, PKC theta was found, based upon the literature, to have an insulin resistant effect only in muscle; however, the present experiments indicate that it might have an important role in the development of insulin resistance in liver. Also, the present experiments indicate that PKC theta is highly expressed in human hepatocytes and thus is an interesting target which induces insulin resistance in skeletal muscle and, as the present experiments have indicated, in liver as well.

### EXAMPLE V

### Effects of PIM2 Reduction on IRS-1 and PKB

One of the promising hits identified and confirmed was the oncogene pim2 (serine/threonine kinase pim2), a member of the family of pim kinases. The X-linked Pim-2 gene is 53% identical to Pim-1 at the amino acid level and shares substrate preference. One of the substrates for pim kinases was recently found to be SOCS-1, and phosphorylation of SOCS-1 by pim2 stabilized the protein in thymocytes.

SOCS-1 is a protein that was found to target IRS1 and IRS2 for ubiquitin-mediated degradation. Adenoviral-mediated expression of SOCS1 in mouse liver dramatically reduced hepatic IRS1 and IRS2 protein levels and caused glucose intolerance in mice.

In accordance with the present invention, the finding that pim2 siRNA prevented IRS-1 degradation by insulin and enhanced insulin-induced PKB phosphorylation made pim2 an interesting target. (See FIG. 6.)

The experiment involving pim2 was carried out as follows:

HepG2 cells were seeded in a 6-well collagen-coated plate. After 24 hours, when they reached 30-40% confluency, they were transfected with pim2 siRNA or control (UC) siRNA. At 48 hours post-transfection, cells were washed and treated with or without 1 uM insulin (Ins). After 18 hours, the cells were lysed and proteins were separated by SDS-PAGE and immunoblotted using anti-IRS-1 or anti-phospho Ser473 antibodies.

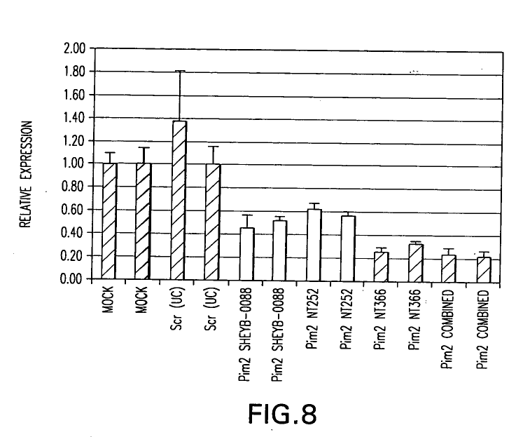

The results were confirmed using two other pim2 specific siRNAs. The knockdown of the pim2 (and pim1 as a control) were tested by RT-PCR. In particular, HepG2 cells were transfected with 100 nM scrambled (universal control), 100 nM individual Pim-2 siRNA or 33 nM each of the three Pim-2 siRNA pooled together for a final concentration of 100 nM. Mock samples received transfection reagent without siRNA. Cells were harvest 48 hrs post-transfection, and RNA was isolated and converted to cDNA. This cDNA was subjected to quantitative PCR with primers specific for the pim2 gene with an internal 28S rRNA control amplification. Each experiment was done in triplicate with the error bars (see FIG. 8) representing standard deviation. Each experiment was normalized to 28S RNA, and the fold difference were calculated to the Mock transfection average. The different pim2 siRNAs tested efficiently downregulated pim2 mRNA, and they did not affect pim1 (data not shown).

Another interesting hit obtained was SNF-1 related kinase (SNRK, NP_{—}060189). SNRK is a 81 kDa protein that is homologous to the AMP kinases and is expressed in tissues such as fat, kidney, liver, testis and thymus. In additional, its expression is increased during adipocyte differentiation. It is involved in ubiquitin ligase binding during proteasomal degradation (Becker et al., Eur J Biochem 235:736-743, 1996; Kertesz et al., Gene 294:13-24, 2002)).

Additionally, it should be noted that a number of kinases involved in the TNF signaling pathway and/or oxidative stress were hits (i.e., met all variables or positive) in the assays or methods described herein. These include, for example, MKK4, MKK7 (i.e., MAP kinase 4 and 7), Jun kinases, oxidative stress responsive 1 kinase and death-associated related kinase 2. Furthermore, kinases involved in the regulation of SOCS and degradation pathway (e.g., Pim, SNF-1 related kinase, etc.) were also hits in the present method.

Table III below summarizes the effects of various kinase-specific siRNAs, discussed above, on IRS-1 degradation and phosphorylation of PKB.

TABLE III

| siRNA | IRS-1 | Phospho-PKB |
|---|---|---|
| S6KB2 | + | + |
| Raptor | + | Corrected + |
| IKK2 | + | + |
| FK506 | No effect | No effect |

### EXAMPLE VI

### Identification of Kinases that Reduce IRS-1 Degradation and Increase Insulin-Induced PKB Phosphorylation

Table IV below illustrates additional kinases that reduce IRS-1 degradation, increase insulin-induced PKB phosphorylation, and thus increase insulin sensitivity. Stimulation of these kinases by a test compound (e.g., an agonist) will positively impact insulin resistance.

The experiments used to identify the properties of the kinases were carried out as follows:

HepG2 cells were plated in a 24 well microtiter plate in MEM and transfected with the different kinase-specific siRNAs (100 nM) or scramble control siRNAs for 48 hrs in duplicate samples. At the end of this period, the cells were washed, serum-starved for 3 hrs, and confluent cells were incubated at 37° C. with low (7 mM) or high (25 mM) glucose in the presence or absence of insulin (1 uM). (Presence of the insulin and glucose allowed the cells to develop an insulin-resistant state.) After 18 hours, the cells were lysed and proteins were separated by SDS/PAGE on 7.5% and 10.0% gels. The electrophoresis was run at 120V. The proteins were then transferred from the gel to nitrocellulose sheets and blocked in 5% milk. The blots were then probed with anti-IRS-1 antibodies (Upstate Biotechnology, Lake Placid, N.Y.) or with phosphoserine 473 antibodies (BD Pharmigen, San Diego, Calif.), according to the manufacturer's recommendations. In particular, IRS-1 protein levels and phosphorylation of PKB were visualized by Western Blotting using the anti-IRS-1 and anti-phospho-PKB antibodies. Decreased detection of IRS-1 and phospho-PKB, or phospho-PKB only, when a siRNA against a specific kinase was used, is indicative of increased insulin resistance. In particular, such results indicate that the kinase per se has a positive role in preventing IRS-1 degradation and increasing PKB phosphorylation resulting in increased insulin sensitivity.

TABLE IV

| | | | Phospho- |
|---|---|---|---|
| siRNA | Kinase information | IRS-1 | PKB |
| AXL | Receptor-tyrosine | Reduced | Reduced |
| | kinase; closely- | | |
| | related to the | | |
| | insulin-receptor | | |
| | family (O'Bryan et al., | | |
| | Mol. Cell. Biol. | | |
| | 11(10): 5016-5031 (1991)). | | |
| Phosphofructokinase | Increased kinase | Reduced | Reduced |
| liver | activity induces | | |
| | increased Glucokinase | | |
| | expression and | | |
| | increases insulin | | |
| | sensitivity in | | |
| | diabetic animals (Wu et | | |
| | al., Endocrinology 145(2): | | |
| | 650-58 (2004)). | | |
| Death-associated | Death-associated | Reduced | Reduced |
| kinase-3 | protein kinase 3 (DAP | | |
| | kinase 3) (DAP-like | | |
| | kinase) | | |
| | (Dlk) (ZIP-kinase) | | |
| Galactokinase 1 | α-D-galactose is | Reduced | Reduced |
| | converted to galactose | | |
| | 1-phosphate via the | | |
| | action of | | |
| | galactokinase (Holden et | | |
| | al., J. Biol. Chem. | | |
| | 278(45): 43885-88 (2003)). | | |
| Fyn-related kinase | Novel family of Src | Reduced | Reduced |
| | kinases | | |

1. A method of identifying a kinase or phosphatase that degrades insulin receptor substrate 1 (IRS-1) and reduces insulin-induced phosphorylation of protein kinase B (PKB) in an insulin-resistant cell, comprising the steps of:
a) transfecting a human hepatoma cell with short interfering ribonucleic acid (siRNA) against said kinase or phosphatase for a time and under conditions sufficient for said cell to incorporate said siRNA into its genome;
b) adding insulin to said resulting cell of step (a) for a time and under conditions sufficient for said cell to become insulin-resistant;
c) lysing said resulting cell of step (b) and separating resulting proteins;
d) determining IRS-1 protein level and phosphorylation of PKB, as compared to that of a cell transfected with control siRNA, an increased amount of IRS-1 and phosphorylated PKB, as compared to said cell transfected with control siRNA, indicating said kinase or phosphatase degrades IRS-1 and decreases phosphorylation of PKB in said insulin-resistant cell.

2. The method of claim 1 wherein said human hepatoma cell is a HepG2 cell.

3. The method of claim 1 wherein said identified kinase is selected from the group consisting of S6 KB2, IKK2, PKC theta, pim 2, pyruvate dehydrogenase, PKC iota, PKC delta, UDP-N-acetylglucosamine-2-epimerimase/N-acetylmannosamine, CaMKI-like protein, DAPK2, casein kinase 1 delta, casein kinase 1 gamma 3, DCAMKL1, SnK Akin kinase, NP_{—}067675, STK10, MAGUK p55 member 2, oxidative-stress responsiveness 1, NP_{—}060189, inositol 1, 3, 4 triphosphate 5-6 kinase, mitogen-activated protein kinase 4, mitogen-activated protein kinase 7, LIM kinase 2 (isoform 2b), phosphorylase kinase alpha 2, salt-inducible protein kinase, Jun kinase 1, 2, dystrophia myotonica protein kinase, CGPK1, MKK6, serine-threonine protein kinase PRP4 homolog, STE-2-like kinase, protein tyrosine kinase 9, P38 delta and adenylate kinase 3 (alpha-like).

4. The method of claim 1 wherein said phosphatase is PTEN.

5. The method of claim 1 wherein said determination of IRS-1 protein level and phosphorylation of PKB is performed by adding anti-IRS-1 and anti-phospho-PKB antibodies to said IRS-1 and phospho-PKB proteins for a time and under conditions sufficient for IRS-1/anti-IRS-1 antibody and phospho-PKB/anti-phospho-PKB antibody complexes to form and determining presence or absence of said complexes as compared to complexes formed from proteins of said cell transfected with control siRNA.

6. A kinase identified according to the method of claim 1.

7. A phosphatase identified according to the method of claim 1.

8. A method of treating a condition in a mammal characterized by diminished uptake and metabolism of glucose comprising the steps of administering to said mammal siRNA against a kinase or phosphatase, wherein said kinase is selected from the group consisting of S6 KB2, IKK2, PKC theta, pim 2, pyruvate dehydrogenase, PKC iota, PKC delta, UDP-N-acetylglucosamine-2-epimerimase/N-acetylmannosamine, CaMKI-like protein, DAPK2, casein kinase 1 delta, casein kinase 1 gamma 3, DCAMKL1, SnK Akin kinase, NP_{—}067675, STK10, MAGUK p55 member 2, oxidative-stress responsiveness 1, NP_{—}060189, inositol 1, 3, 4 triphosphate 5-6 kinase, mitogen-activated protein kinase 4, mitogen-activated protein kinase 7, LIM kinase 2 (isoform 2b), phosphorylase kinase alpha 2, salt-inducible protein kinase, Jun kinase 1, 2, dystrophia myotonica protein kinase, CGPK1, MKK6, serine-threonine protein kinase PRP4 homolog, STE-2-like kinase, protein tyrosine kinase 9, P38 delta and adenylate kinase 3 (alpha-like) and said phosphatase is PTEN, in an amount sufficient to effect said treatment.

9. The method of claim 8 wherein said mammal is selected from the group consisting of a human, a domesticated animal and a non-domesticated animal.

10. The method of claim 8 wherein said condition is diabetes.

11. The method of claim 10 wherein said diabetes is Type 2 diabetes.

12. A method of identifying a compound which inhibits or negatively alters the function of a kinase, wherein said kinase causes IRS-1 degradation and reduces insulin signaling in an insulin-resistant cell, comprising contacting said test compound with said kinase for a time and under conditions sufficient for complexes to form between said test compound and said kinase, presence of said complexes indicating a compound which inhibits or negatively alters said function of said kinase.

13. A method of identifying a compound which inhibits or negatively alters the function of a phosphatase, wherein said phosphatase causes IRS-1 degradation and reduces insulin signaling in an insulin-resistant cell, comprising contacting said test compound with said phosphatase for a time and under conditions sufficient for complexes to form between said test compound and said phosphatase, presence of said complexes indicating a compound which inhibits or negatively alters said function of said phosphatase.

14. A method of reducing or inhibiting IRS-1 degradation and increasing insulin-induced phosphorylation of PKB in a mammal in need of said reduction or inhibition of IRS-1 degradation and increased insulin-induced phosphorylation of PKB comprising administering to said mammal an siRNA against a kinase, wherein said kinase is selected from the group consisting of S6 KB2, IKK2, PKC theta, pim 2, pyruvate dehydrogenase, PKC iota, PKC delta, UDP-N-acetylglucosamine-2-epimerimase/N-acetylmannosamine, CaMKI-like protein, DAPK2, casein kinase 1 delta, casein kinase 1 gamma 3, DCAMKL1, SnK Akin kinase, NP_{—}067675, STK10, MAGUK p55 member 2, oxidative-stress responsiveness 1, NP_{—}060189, inositol 1, 3, 4 triphosphate 5-6 kinase, mitogen-activated protein kinase 4, mitogen-activated protein kinase 7, LIM kinase 2 (isoform 2b), phosphorylase kinase alpha 2, salt-inducible protein kinase, Jun kinase 1, 2, dystrophia myotonica protein kinase, CGPK1, MKK6, serine-threonine protein kinase PRP4 homolog, STE-2-like kinase, protein tyrosine kinase 9, P38 delta and adenylate kinase 3 (alpha-like) in an amount sufficient to effect said reduction or inhibition of IRS-1 degradation and increased insulin-induced phosphorylation of PKB.

15. The method of claim 14 wherein said mammal is selected from the group consisting of a human, a domesticated animal and a non-domesticated animal.

16. A method of reducing or inhibiting IRS-1 degradation and increasing insulin-induced phosphorylation of PKB in a mammal in need of said reduction or inhibition of IRS-1 degradation and increased insulin-induced phosphorylation of PKB comprising administering to said mammal an siRNA against a phosphatase, wherein said phosphatase is PTEN, in an amount sufficient to effect said reduction or inhibition of IRS-1 degradation and increased insulin-induced phosphorylation of PKB.

17. The method of claim 16 wherein said mammal is selected from the group consisting of a human, a domesticated animal and a non-domesticated animal.

18. A method of decreasing IRS-1 degradation and increasing insulin-induced phosphorylation of PKB in a mammal in need of said decrease of IRS-1 degradation and increased insulin-induced phosphorylation of PKB comprising administering to said mammal an agonist of the kinase, wherein said kinase is selected from the group consisting of AXL, liver phosphofructokinase, death-associated kinase-3, galactokinase 1 and fyn-related kinase, in an amount sufficient to effect said reduced IRS-1 degradation and increased insulin-induced phosphorylation of PKB.

19. The method of claim 18 wherein said mammal is selected from the group consisting of a human, a domesticated animal and a non-domesticated animal.

20. A method of identifying a compound which increases activity of a kinase, wherein activity of said kinase prevents IRS-1 degradation, comprising contacting said test compound with said kinase for a time and under conditions sufficient for complexes to form between said test compound and said kinase, presence of said complexes indicating a compound which increases activity of said kinase.

21. The method of claim 20 wherein said kinase is selected from the group consisting of AXL, liver phosphofructokinase, death-associated kinase-3, galactokinase 1 and fyn-related kinase.
